(19) United States
(12) Patent Application Publication
(10) Pub. No.: US 2012/0003339 A1
(43) Pub. Date: Jan. 5, 2012

(54) COMPOSITIONS AND METHODS FOR CONTROLLING BLOOD GLUCOSE LEVELS

(76) Inventor: Pompeo MINACAPELLI, Mississauga (CA)

(21) Appl. No.: 12/846,184

(22) Filed: Jul. 29, 2010

Related U.S. Application Data
(60) Provisional application No. 61/361,049, filed on Jul. 2, 2010.

(51) Int. Cl. A61K 36/28; A61P 3/10

(52) U.S. Cl. 424/764

(57) ABSTRACT

Disclosed herein are methods and compositions for controlling the blood glucose levels of a patient with diabetes, comprising administering a composition comprising an effective amount of Gudmar, Kalijiri, Kariyatu, and Neempan.

### RELATED APPLICATIONS

This application claims the priority benefit of U.S. Provisional Application No. 61/361,049 filed Jul. 2, 2010 the entirety of which is incorporated herein by reference.

### BACKGROUND

Diabetes mellitus (diabetes) is a chronic disease marked by high levels of glucose in the blood. It is associated with an impaired glucose cycle, altering metabolism. In 2009, 11.3% of adults in the United States (or about 26 million Americans) were reported to have diabetes, and the rate is projected to increase to 15% (or more than 37 million Americans) by the end of 2015. The incidence of diabetes has been reported to have doubled in the past 30 years.

Diabetes has become a major health problem worldwide. This, coupled with the chronicity of the disease, relate to an increasing burden on health care facilities and an increasing number of hospital admissions of patients suffering from diabetes. Admissions are mostly related to diabetes itself, but the frequency of admissions for problems not related to diabetes is increasing, as the prevalence of diabetes increases in the population. Proper inpatient glycemic management is important for improving patient outcome and for reducing the risk of inpatient complications. Management of this disease may include carefully managing diet, exercising, taking oral diabetes medication, using some form of insulin, maintaining proper circulation in extremities. Although there are numerous management strategies and numerous medications available to control the blood glucose levels in patients with diabetes, a large number of diabetic patients experience difficulty controlling blood glucose levels within the normal range, which can cause various complications such as cardiovascular disease, stroke, blindness, nerve and renal damage and inflammatory disorders. Moreover, there are numerous side effects associated with anti-diabetic medications, which can include but are not limited to hypoglycemia, hyperglycemia, blurred vision, dizziness, fatigue, sweating, clumsy or jerky movements, severe migraines, upper respiratory tract infection, headache, back pain, fatigue, sinusitis, nausea, vomiting, gas, bloating, diarrhea, constipation, loss of appetite, dizziness, weight gain, liver disease, swelling in legs and ankles, and mild to moderate edema, which can lead to heart failure.

For example, type 2 diabetes, which represents 80% of all diabetes in the United States and affects almost 18 million Americans, is often initially managed by increasing exercise and dietary modification. As the condition progresses, medications are typically needed. The medications include hormone/peptide analog injections, such as insulin, pramlintide (brand name Symlin), and exenatide (brand name Byetta), or anti-diabetic drugs that lower blood glucose levels. Currently, all the anti-diabetic drugs sold in the United States belong to six classes of compounds: sulfonylureas, meglitinides, biguanides (metformin), thiazolidinediones (rosiglitazone), alpha-glucosidase inhibitors, and DPP-4 inhibitors. However, a significant number of patients treated with these anti-diabetic drugs still have difficulty controlling blood glucose levels within the normal range, while others may experience one or more of the above-mentioned side effects associated with these anti-diabetic drugs.

Accordingly, a need remains for new effective and safe medications that are capable of controlling blood glucose levels in diabetic patients without the severe side effects associated with traditional anti-diabetic drugs.

### SUMMARY

The present invention relates to the unexpected discovery that particular combinations of natural herbal compositions can effectively control the blood glucose levels of a subject. In one embodiment, the subject is a diabetic patient and the combination of the present invention controls the blood glucose levels of the diabetic patient without the side effects associated with traditional anti-diabetic medications. The natural herbs in the compositions of the present invention include Gudmar, Kalijiri, Kariyatu, and Neempan.

An effective daily standardized amount of each herb ranges from about 5 mg to 500 mg, alternatively about 25 mg to 200 mg, alternatively about 50 mg to 100 mg, alternatively about 60 mg to 90 mg, in another alternative 70 mg to 80 mg of Gudmar, Kalijiri, Kariyatu, and Neempan independently. For example, the daily dose of natural herbs can include 500 mg of Gudmar, 5 mg of Kalijiri, 100 mg of Kariyatu, and 325 mg of Neempan. In one embodiment, the daily dose includes 75 mg of Gudmar, 75 mg of Kalijiri, 75 mg of Kariyatu, and 75 mg of Neempan. In one embodiment, each ingredient is taken separately in the form of a tablet. In another embodiment, two, three, or four of the herbs are combined in a single tablet. The daily dose can be administered in one, two, three, four, or five doses.

The present invention further provides a method of controlling blood glucose levels in a subject by administering to the patient an effective daily amount of each of the natural herbs Gudmar, Kalijiri, Kariyatu, and Neempan. One embodiment is directed to a daily dose of 75 mg Gudmar, 75 mg Kalijiri, 75 mg Kariyatu, and 75 mg Neempan. In a particular embodiment, each ingredient is administered in three equal doses of 25 mg, taken about eight hours apart. In a particular embodiment, each ingredient is administered in two equal doses of 37.5 mg, taken about 12 hours apart.

In another aspect, the present invention provides a kit for controlling blood glucose levels in a subject, comprising Gudmar, Kalijiri, Kariyatu and Neempan, each separately in the form of a tablet. In an alternative embodiment, two, three, or four herbs are combined in a single tablet. In one embodiment, the kit comprises sufficient tablets for controlling the blood glucose level of a subject for 30 days. In alternative embodiments, the kit comprises sufficient tablets for controlling the blood glucose level of a subject for 7, 14, 21, or 28 days. In yet other alternative embodiments, the kit comprises sufficient tablets for controlling the blood glucose level of a subject for 2, 3, 4, 5, 6, 9, or 12 months.

The subjects who can benefit from the herbal composition of the present invention include, but are not limited to, normal subjects, subjects with blood glucose levels at the high end of the normal range (i.e., pre-diabetic), subjects with hyperglycemia, subjects with diabetes (including Type 1A, Type 1B, and Type 2).

In another aspect, the method further comprises administering to the diabetic patient an effective amount of a traditional antidiabetic drug. In one embodiment, the traditional anti-diabetic drug is rosiglitazone or metformin. In another embodiment, the traditional anti-diabetic drug is insulin.

In another embodiment, the method comprises initially administering to the diabetic patient the composition of the present invention along with an effective amount of a traditional anti-diabetic drug and, as the blood glucose levels return to normal, the amounts of a traditional anti-diabetic drug are progressively reduced. In some diabetic patients, the amounts of a traditional anti-diabetic drug are reduced to zero and the blood glucose levels are controlled solely with the herbal composition of the present invention.

### DETAILED DESCRIPTION

Diabetes comprises a group of common metabolic disorders that share the phenotype of hyperglycemia. Several distinct types of diabetes exist and are caused by a complex interaction of genetics, environmental factors, and life-style choices. Depending on the etiology of the diabetes, factors contributing to hyperglycemia may include reduced insulin secretion, decreased glucose usage, and increased glucose production. The metabolic dysregulation associated with diabetes causes secondary pathophysiologic changes in multiple organ systems that impose a tremendous burden on the individual with diabetes and on the health care system. In the United States, diabetes is the leading cause of end-stage renal disease, nontraumatic lower extremity amputations, and adult blindness. With an increasing incidence worldwide, diabetes will likely continue to be a leading cause of morbidity and mortality for the foreseeable future.

Recent advances in the understanding of the etiology and pathogenesis of diabetes have led to a revised classification (Table 1). Although all forms of diabetes are characterized by hyperglycemia, the pathogenic mechanisms by which hyperglycemia arises differ widely. Some forms of diabetes are characterized by an absolute insulin deficiency or a genetic defect leading to defective insulin secretion, whereas other forms share insulin resistance as their underlying etiology. Recent changes in classification reflect an effort to classify diabetes on the basis of the pathogenic process that leads to hyperglycemia, as opposed to criteria such as age of onset or type of therapy.

The two broad categories of diabetes are designated type 1 and type 2. Type 1A diabetes results from autoimmune beta cell destruction, which usually leads to insulin deficiency. Type 1B diabetes is also characterized by insulin deficiency as well as a tendency to develop ketosis. However, individuals with type 1B diabetes lack immunologic markers indicative of an autoimmune destructive process of the beta cells. The mechanisms leading to beta cell destruction in these patients are unknown. Relatively few patients with type 1 diabetes fall into the type 1B idiopathic category; many of these individuals are either African-American or Asian in heritage.

Type 2 diabetes is a heterogeneous group of disorders usually characterized by variable degrees of insulin resistance, impaired insulin secretion, and increased glucose production. Distinct genetic and metabolic defects in insulin action and/or secretion give rise to the common phenotype of hyperglycemia in type 2 diabetes.

### Herb-Containing Compositions of the Invention

One object of the present invention is to provide new and useful herb-containing compositions and methods for controlling blood glucose levels in a patient with diabetes. The composition of the present invention is comprised of the following four (4) natural herbs:

### Gudmar

Gudmar (also known as Gurmar, Gymnema Sylvestre, Gymnema, Gurmarbooti, Periploca of the woods, Meshasringi, Kavali, Sarpadarushtrika, Wakandi, Shiru-kurunja, Bodaparta, Putla-podra, Chakkarakolli, Parpatrah, Cherukurinja, Meshasringi, Chhota-Dudhilata, Merasingi) is a woody climbing plant that grows in the tropical forests of central and southern India. The herb's active ingredient, gymnemic acid, is extracted from leaves and roots. The fresh leaves when chewed have the remarkable property of paralyzing the sense of taste for sweet and bitter substance for some time, which explains the Hindi name gurmar which means “destroyer of sugar.” The herb has often promoted as an appetite suppressant and weight-loss agent. It has been reported to lower blood sugar level and is good for the treatment of both types of diabetes.

### Kalijiri

Kalijiri (also known as Centratherum Anthelminticum, jangali jiri, banjira, somraj, somraji, kalenjiri, jangali jiri, kalen jiri) is a common weed in India that has a much branched stem from 2 to 3 feet in height and numerous, lance-shaped leaves, the lower ones 1 to 3 inches in length and the upper ones much smaller. The greenish flowers are produced from July to September in closely crowded spikes mixed with leaves and are followed by small, green, roundish fruits, each of which contains a very small black seed. Kalijiri is extracted from the seeds.

### Kariyatu

Kariyatu (also known as Andrographis paniculata, Maha-tita, Bhul-neem, Swertia Chirata, Chirayata, Sambiloto, the Creat, Kariyat, Nelavepu, Kiriyattu, Hempedu Bumi, Pokok Akar Cerita) is an erect annual herb indigenous to temperate Himalayas at altitudes above 1000 meter from Kashmir but also found in other parts. It is widely cultivated in southern Asia, where it is used to treat infections and some diseases. Mostly the leaves and roots were used for medicinal purposes. The plant extract contains the bitter principles amarogentine, and amarowserin. This herb has been reported to have numerous therapeutic actions: antibacterial, analgesic, anti-inflammatory, antioxidant, anti-carcinogenic, anti-pyretic, anti-thrombic, anti-viral, digestive, hypoglycemic, blood purifier, vermicidal and adaptogen (i.e., helps to normalize a physical function, depending on what the individual needs). It has also been reported to exhibit anti-hepatotoxic and anti-ulcerogenic activities. Studies show this herb is a bitter tonic and is an excellent remedy for a weak stomach, especially when this gives rise to nausea, indigestion and bloating. It has also been reported to protect the liver. The plant is an excellent drug for sporadic fevers, skin diseases intestinal worms, bronchial asthma, burning of the body. It is also used in the liquor industry as a bitter ingredient.

### Neempan

Neempan (also known as Azadirachita Indica, Indian Lilac, Bead Tree, Holy Tree, Margosa Tree, Neem, Nim, Persian Lilac, Pride of China, Ravipriya, Veppu) is derived from the Nccm tree, a fast growing, umbrella shaped, evergreen tree belonging to the mahogany family. The Neem tree is found in every part of India and considered an excellent herb that believes to cure almost hundred diseases. Each part has a different therapeutic value. For example, the root bark is reported to be an astringent, tonic; the bark of the tree is reported to be an astringent, antiviral, bitter, tonic, and the leaves are reported to have antiviral properties. Since Neem is very bitter and diabetes is a disease of excess sweetness; it was often used to treat diabetes in Ayurveda. Neem formulations have also been reported to be useful in arthritis, blood (purifies and detoxifies), bronchitis, cough, drowsiness, eczema, fever, jaundice, and malaria. Neem has over 4500 hundred years of use in its native India, where it is referred to as the “village pharmacy”.

The term “an effective amount” refers to the amount (dose) when administered to the patient which results in beneficial or desired results, including clinical results, e.g., reduces the patient's blood glucose levels to the normal range.

Generally, an effective amount of an ingredient of the invention varies depending upon various factors, such as the given ingredient, the pharmaceutical formulation, the route of administration, the type of disease or disorder, the identity of the patient or host being treated, and the like, but can nevertheless be routinely determined by one skilled in the art. An effective amount of a compound of the present invention may be readily determined by one of ordinary skill by routine methods known in the art. Table 2 provides the ranges of effective daily doses Gudmar, Kalijiri, Kariyatu, and Neempan.

As used herein, the term “controlling blood glucose levels” refers to medications that maintain a patient's blood glucose levels within the normal range. A fasting plasma glucose level that is less than 6.1 mmol/L (110 mg/dL) and a 2 hour postprandial plasma glucose level that is less than 7.8 mmol/L (140 mg/dL) are considered to be the normal range. However, a fasting plasma glucose level that is greater than 7.0 mmol/L (126 mg/dL) and a 2 hour postprandial plasma glucose level that is greater than 11.1 mmol/L (200 mg/dL) meet the threshold for the diagnosis of diabetes. Patients with diabetes also need to have their hemoglobin A1c (HbA1c) level checked every 3-6 months. The HbA1c is a measure of average blood glucose during the previous 2-3 months. It is a very helpful way to determine how well treatment is working. An HbA1c level of 6% to 7% is considered a desirable goal for patients.

Moreover, a “treatment” regime of a patient with an effective amount of the ingredients of the present invention may consist of a single administration, or alternatively comprise a series of applications. For example, the ingredients of the present invention may be administered at one, two, or three times a day for a period of one, two, or three weeks, or one or more months. The length of the treatment period depends on a variety of factors, such as the severity of the disease, the age of the patient, the concentration and the activity of the ingredients of the present invention, or a combination thereof. It will also be appreciated that the effective dosage of the ingredients used for the treatment may increase or decrease over the course of a particular treatment regime. Changes in dosage may result and become apparent by standard diagnostic assays known in the art. In some instances, chronic administration may be required.

The ingredients of the invention can be administered to a patient in a variety of forms depending on the selected route of administration, as will be understood by those skilled in the art. The ingredients of the invention may be administered, for example, by oral administration and the herbal compositions formulated accordingly. Typically, for oral administration, a compound of the invention may be incorporated with excipient/pharmaceutically-acceptable carriers and used in the form of ingestible tablets, buccal tablets, troches, capsules, elixirs, suspensions, syrups, wafers, and the like. In one embodiment, the four herbal ingredients are taken individually. Alternatively, two, three, or four of the herbal ingredients are combined in a single mixture of the above forms.

An excipient is an inactive substance used as a carrier for the active ingredients of a medication. In some cases, an active substance may not be easily administered and absorbed by the human body. In such cases the substance in question can be mixed with an excipient. Excipients can also be used to bulk up formulations that contain very potent active ingredients, to allow for convenient and accurate dosage. In addition to their use in the single dosage quantity, excipients can be used in the manufacturing process to aid in the handling of the active substance concerned. Depending on the route of administration, and form of medication, different excipients may be used, as determined by one of skill in the art.

Pharmaceutically-acceptable carriers can include carriers suitable for oral administration, for example, by allowing diffusion of active agents from capsules or tablets. Such carriers are known to those of skill in the art and can be selected according to the mode of administration.

Depending on the severity of the disease, diabetes is initially treated by adjustments in diet and exercise, and by weight loss. Patients with more advanced conditions will need medications. In one embodiment, the ingredients of the present invention are administered to the patient in combination with a traditional anti-diabetic drug. Traditional anti-diabetic drugs belong to six classes of compounds: sulfonylureas, meglitinides, biguanides (metformin), thiazolidinedioncs (rosiglitazone), alpha-glucosidase inhibitors, and DPP-4 inhibitors, which are administered orally. In a particular embodiment, the traditional anti-diabetic drug is rosiglitazone or metformin.

In a particular embodiment, the ingredients of the present invention are administered to the patient in combination with an effective amount of insulin to maintain normal or near normal glucose levels.

Typical total daily dosage of insulin is 0.6 U/kg, with best timing and total amounts depending on diet (composition, amount, and timing) as well the degree of insulin resistance. More complicated estimations to guide initial dosage of insulin are:
For men,

[(fasting plasma glucose [mmol/L]−5)×2]×(weight [kg]÷(14.3×height [m])−height [m])
For women,

[(fasting plasma glucose [mmol/L]−5)×2]×(weight [kg]÷(13.2×height [m])−height [m])

The initial insulin regimen are often chosen based on the patient's blood glucose profile. Initially, adding nightly insulin to patients failing oral medications may be best. Nightly insulin combines better with metformin than with sulfonylureas. The initial dose of nightly insulin (measured in IU/d) should be equal to the fasting blood glucose level (measured in mmol/L).

When nightly insulin is insufficient, choices include: premixed insulin with a fixed ratio of short and intermediate acting insulin; long acting insulins such as insulin glargine and insulin detemir; and insulin pump therapy.

### EXAMPLES

The following examples demonstrate the efficacy of the herbal medication of the present invention for controlling blood glucose levels in patients with diabetes.

### Example I

A 76 year old male subject was diagnosed with type 2 diabetes at approximately age 70. His blood glucose levels were 17 to 19 mmol/L. The subject was first prescribed Avandia, which did not reduce his blood glucose levels to the normal range. Accordingly, Avandia was then replaced with Metformin. With these treatments, the subject's blood glucose levels were reduced to between 14 and 15 mmol/L. The best level achieved was 12 mmol/L, which is not within the normal range.

In addition to the inability of properly controlling the blood glucose levels, the traditional anti-diabetic medicines induced numerous adverse side effects in the subjects. He had blurred vision, fatigue, severe migraines and constipation. He also had trouble controlling his weight. He was experiencing shaking on a daily basis, especially his right arm and was certain he had Parkinson's. He felt his balance was off and felt quite wobbly. He had trouble sleeping and often would wake in the middle of the night and sometimes needed to eat.

The subject started taking 25 mg each of Gudmar, Kalijiri, Kariyatu, and Neempan tablets three times a day and stopped taking the traditional anti-diabetic medicines. Within 7-10 days of taking the formulation of the present invention, the subject's blood glucose levels quickly dropped to within the normal range and the side effects disappeared. The subject had no blurred vision, lots of energy, regular bowel movements, and is controlling his weight. He was not experiencing any of the shaking that he was before and slept extremely well, rising well rested. The subject seldom had a migraine and was able to eat a full diet including sweets in moderation. His doctor has monitored and continues to monitor his condition on a regular basis and is amazed at his results.

These results indicate that the composition and method of the present invention are effective in helping a diabetic subject control his blood glucose level without the side effects of traditional anti-diabetic medicines.

Interestingly, on several occasions, the subject has stopped taking the herbal medication for a certain period of time. In each instances, the subject's blood glucose levels gradually increased well above normal levels. Once the subject started taking the herbal medication again, his blood glucose gradually dropped and returned to normal within 14 days.

In one of these occasions, the subject stopped taking the herbal medication for 14 days. During the 14 days, his blood glucose levels gradually increased, and by day 14, his blood glucose levels were at 27 mmol/L. Upon resuming the herbal medication, his blood glucose levels dropped back to 17 mmol/L on the first day, then to 14, 13, and 12 mmol/L in subsequent days. The subject's blood glucose levels gradually reduced to 7-7.5 mmol/L in 10 days. During the herbal medication period, the subject had a normal diet with no restrictions, except that his diet contained no added sugar such as cake, pie, or soft drinks, etc.

These results indicate that the composition and method of the present invention were the critical element in helping this diabetic subject control his blood glucose level.

### Example II

A 51 year old male subject had been diagnosed with type 2 diabetes at age 50. His fasting glucose reading was 11.6 mmol/L and A1c was 8.9%. Due to arthritis in his knee, exercise was not an option. Further, his diet did not materially change since his wife was diabetic for a while. He was prescribed 500 mg of Ratio-Metformin once a day. While the medication did reduce his blood glucose levels, it left him feeling confused, “in a fog.” As a result, he was sent to see an endocrinologist, at the Diabetic Clinic of the local hospital. According to the endocrinologist, the subject's morning readings (fasting blood glucose levels) were in the range from 8.1 to 10.8 mmol/L, and his two-hour after eating readings were 10 to 13.8 mmol/L.

Due to the adverse side effects of Metformin, the subject decided to stop taking the prescribed daily Metformin and replace it with the composition and method of the present invention. The adverse side effects disappeared as soon as the subject stopped taking Metformin. The subject has now taken three cycles of the composition of the present invention.

The first cycle was about three months in length. The subject took a total of about 180 doses of 25 mg each of Gudmar, Kalijiri, Kariyatu, and Neempan tablets 3 times a day for about 4 weeks, followed by 2 times a day for about 4 weeks, and followed by once a day until finished. Within a week that the subject was treated with the method of the invention, his morning readings were in the 4 to 6.5 mmol/L range and his two hours after eating range were well below 9 mmol/L. It should be noted that, for three weeks during this period, the patient was on vacation in France, enjoying fine French food and wine. A month after finishing the first cycle, the subject took a second fasting sugar test and a A1c test, the readings were 6.6 mmol/L and 6%, respectively.

The subject was treated with three cycles of the composition and method of the present invention. After the patient ended the first cycle, his blood glucose levels remained fairly normal but was slowly increasing. When his morning readings were above 7 mmol/L; which was three months after the end of the first cycle, the subject started the second cycle of herbal treatments. During the second cycle, the subject took a total of about 90 doses of 25 mg each of Gudmar, Kalijiri, Kariyatu, and Neempan tablets 3 times a day for about a month. Once he started the second cycle, the subject experienced a drop in blood glucose levels. His morning readings were in the range of 4.3 to 6.5 mmol/L, and his two hour after eating readings were in the range of 6 to 8.5 mmol/L.

The third cycle began about four months after the second cycle ended when his morning readings started to exceed 7.4 mmol/L. During the third cycle, the subject took a total of about 90 doses of 25 mg each of Gudmar, Kalijiri, Kariyatu, and Neempan tablets two times a day for 45 days. Again, the subject experienced a drop in blood glucose levels. At the end of the third cycle, the subject's morning reading was 4.8 mmol/L.

### Example III

A 38 year old male subject weighed 380 lb was diagnosed with type 2 diabetes and was considered morbidly obese. The subject was prescribed Metformin. With traditional medication his blood glucose levels were at about 19 mmol/L, well above the normal range. Although the subject was considered for gastric bypass surgery, his medical doctors would not operate because his blood glucose could not be controlled.

The subject started taking 25 mg each of Gudmar, Kalijiri, Kariyatu, and Neempan tablets three times a day. Two weeks after the subject started the taking the herbal composition of the invention, the subject's blood glucose levels had dropped to 5.5 mmol/L, within the normal range. As a result, the subject was able to proceed with the surgery.

These three examples indicate that the composition and method of the present invention are effective in helping a diabetic subject control his blood glucose level without the side effects of traditional anti-diabetic medicines.

### EQUIVALENTS

This invention has been described in terms of specific embodiments set forth in detail herein, but it should be understood that these are by way of illustration and the invention is not necessarily limited thereto. Modifications and variations will be apparent from the disclosure and may be resorted to without departing from the spirit of the invention as those of skill in the art will readily understand. Accordingly, such variations and modifications are considered to be within the purview and scope of the invention and the following claims.

TABLE 1
Etiologic Classification of Diabetes Mellitus¹

| I. | Type 1 diabetes (β-cell destruction, usually leading to absolute insulin deficiency) | |
|---|---|---|
| | A. | Immune-mediated |
| | B. | Idiopathic |
| II. | Type 2 diabetes (may range from predominantly insulin resistance with relative insulin | |
| | deficiency to a predominantly insulin secretory defect with insulin resistance). | |
| III. | Other specific types of diabetes | |
| | A. | Genetic defects of function characterized by mutations in: |

| | |
|---|---|
| 1. | Hepatocyte nuclear transcription factor (HNF) 4α (MODY 1)* |
| 2. | Glucokinase (MODY 2) |
| 3. | HNF-1α (MODY 3) |
| 4. | Insulin promoter factor (IPF) 1 (MODY 4) |
| 5. | HNF-1β (MODY 5) |
| 6. | Mitochondrial DNA |
| 7. | Proinsulin or insulin conversion |

| | |
|---|---|
| B. | Genetic defects of in insulin action |

| | |
|---|---|
| 1. | Type A insulin resistance |
| 2. | Leprechaunism |
| 3. | Rabson-Mendenhall syndrome |
| 4. | Lipoatrophic diabetes |

| | |
|---|---|
| C. | Diseases of the exocrine pancreas: pancreatitis, pancreatectomy, neoplasia, cystic |
| | fibrosis, hemochromatosis, fibrocalculous pancreatopathy |
| D. | Endocrinopathies: acromegaly, Cushing's syndrome, glucagonoma, pheochromocytoma, |
| | hyperthyroidism, somatostatinoma, aldosteronoma |
| E. | Drug or chemical-induced: Vacor, pentamidine, nicotinic acid, glucocorticoids, thyroid |
| | hormone, diazoxide, β-adrenergic agonists, thiazides, phenytoin, α-interferon, protease |
| | inhibitors, clozapine, beta blockers |
| F. | Infections: congenital rubella, cytomegalovirus, coxsackle |
| G. | Uncommon forms of immune-mediated diabetes: “stiff-man syndrome, anti-insulin |
| | receptor antibodies |
| H. | Other genetic syndromes associated with diabetes: Down's syndrome, Klinefelter's |
| | syndrome, Turner's syndrome, Wolfram's syndrome, Friedreich's ataxia, Huntington's |
| | chorea, Laurence-Moon-Biedl syndrome, myotonic dystrophy, porphyria, Prader-Willi |
| | syndrome |

| | |
|---|---|
| IV. | Gestational diabetes mellitus (GDM) |

¹Harrison's Principles of Internal Medicine, 15^{th} edition.
*MODY, maturity onset of diabetes of the young

TABLE 2
Standardized Herbal Composition of the Present Invention

| | Range A | Range B | Range C | Range D | Range E |
|---|---|---|---|---|---|
| Components | (mg/day) | (mg/day) | (mg/day) | (mg/day) | (mg/day) |
| Gudmar | 5-500 | 25-200 | 50-100 | 60-90 | 70-80 |
| Kalljiri | 5-500 | 25-200 | 50-100 | 60-90 | 70-80 |
| Kariyatu | 5-500 | 25-200 | 50-100 | 60-90 | 70-80 |
| Neempan | 5-500 | 25-200 | 50-100 | 60-90 | 70-80 |

What is claimed is:

1. A composition for controlling blood glucose levels, comprising the following herbal ingredients:
(a) 5-500 mg of Gudmar,
(b) 5-500 mg of Kalijiri,
(c) 5-500 mg of Kariyatu, and
(d) 5-500 mg of Neempan.

2. The composition of claim 1, wherein each herbal ingredient is separately in the form of a tablet.

3. The composition of claim 1, wherein the four herbal ingredients are combined in a single tablet.

4. A kit for controlling blood glucose levels, comprising Gudmar, Kalijiri, Kariyatu and Neempan, each separately in the form of a tablet.

5. A method of controlling blood glucose levels in a subject, comprising administering to the patient a daily dose of the following herbal ingredients:
(a) 50-100 mg of Gudmar,
(b) 50-100 mg of Kalijiri,
(c) 50-100 mg of Kariyatu, and
(d) 50-100 mg of Neempan.

6. The method of claim 5, wherein each herbal ingredient is administered in three equal doses.

7. The method of claim 5, wherein each herbal ingredient is administered in two equal doses.

8. The method of claim 6, wherein each herbal ingredient is administered separately in the form of a tablet.

9. The method of claim 7, wherein each herbal ingredient is administered separately in the form of a tablet.

10. The method of claim 6, wherein the four ingredients are combined in a single tablet.

11. The method of claim 7, wherein the four ingredients are combined in a single tablet.

12. The method of claim 5, wherein the subject has hyperglycemia.

13. The method of claim 5, wherein the subject has diabetes.

14. The method of claim 13, wherein the subject has Type 1 diabetes.

15. The method of claim 14, wherein the subject has Type 1A diabetes.

16. The method of claim 14, wherein the subject has Type 1B diabetes.

17. The method of claim 13, wherein the subject has Type 2 diabetes.
